(19) United States
(12) Patent Application Publication
(10) Pub. No.: US 2019/0070140 A1
(43) Pub. Date: Mar. 7, 2019

(54) COMPOSITION COMPRISING PROTECTIN DX AS ACTIVE INGREDIENT FOR PREVENTING OR TREATING HYPERLIPIDEMIA OR FATTY LIVER DISEASE

(71) Applicant: Solvit P&F, Seoul (KR)

(72) Inventors: Yuan Lu Sun, Seoul (KR); Ji Hoon Jeong, Gyeonggi-do (KR); Tae Woo Jung, Seoul (KR)

(21) Appl. No.: 16/115,983

(22) Filed: Aug. 29, 2018

(30) Foreign Application Priority Data
Sep. 1, 2017 (KR) 10-2017-0112118

(51) Int. Cl. A61K 31/202; A61P 1/16; A61P 3/06; A61P 3/04

(52) U.S. Cl. CPC A61K 31/202; A61P 3/04; A61P 3/06; A61P 1/16

(57) ABSTRACT

The present invention relates to use of a composition comprising protectin DX of Formula 1 as an effective ingredient for treating, preventing or ameliorating hyperlipidemia or fatty liver disease and/or protecting the liver.

### CROSS-REFERENCE TO RELATED APPLICATIONS

This application claims priority from and the benefit of Korean Patent Application No. 10-2017-0112118, filed on Sep. 1, 2017 with the Korean Intellectual Property Office. The disclosures of the priority application are herein incorporated by reference in its entirety.

### BACKGROUND FIELD

The present invention relates to a composition for prevention or treatment of hyperlipidemia or fatty liver disease, including protectin DX as an active ingredient and, more particularly, to a pharmaceutical composition for prevention or treatment of hyperlipidemia or fatty liver disease, which includes protectin DX represented by Formula 1 or pharmaceutically acceptable salts thereof as an active ingredient.

### DISCUSSION OF THE BACKGROUND

Recently, as the overall nutritional status of people is improved and adult diseases have increased, the number of patients suffering from hyperlipidemia tends to increase. Hyperlipidemia refers to a condition where an amount of lipid components in blood has been abnormally increased. An increase in cholesterol and triglyceride in blood serum is considered as the most general cause of hyperlipidemia, and excessive fat and/or lipid accumulation may cause blood circulation disorders and micro-circulatory failure.

Further, similar to hyperlipidemia, the number of patients suffering from fatty liver disease has also tended to increase. Fatty liver is a disease caused by abnormal accumulation of fat (such as triglyceride) in the liver, thus leading to a hindrance to liver function. The initial pathological condition of fatty liver disease is a simple fat liver where fat deposit only is recognized in liver cells. Thereafter, it is known that the pathological condition is progressing to steatohepatitis (including hepatic fibrosis), and also to cirrhosis or hepatocellular carcinoma. Typically, causes of fat or lipid deposition in the liver may include, for example, alcohol intake, obesity, diabetes, lipid metabolism disorder, that is, dyslipidemia, medicine (steroid, tetracycline, etc.), Cushing's syndrome, poisoning (white phosphorus, etc.), serious nutritional disorder, or the like.

Causes of fatty liver disease are generally divided into alcoholic and non-alcoholic. The former is called alcoholic fatty liver disease (or alcoholic liver disorder), while the latter is called non-alcoholic fatty liver disease (NAFLD).

Alcoholic fatty liver disease progresses from the initial simple fatty liver to steatohepatitis, cirrhosis, etc. On the other hand, non-alcoholic fatty liver disease is considered not to involve progress of pathological condition while remaining in the simple fatty liver status, however, it has recently been discovered that even the non-alcoholic fatty liver disease sometimes exhibits a pathological condition progressing from a simple fatty liver to steatohepatitis or cirrhosis.

Non-alcoholic fatty liver disease (NAFLD) refers to a case where fatty change or steatosis and/or lobular hepatitis or steatohepatitis are observed as findings specific to alcoholic hepatitis in liver biopsy although a specific alcohol intake history determined to be harmful to the liver does not exist. Pathological findings of the liver may exhibit diverse spectra such as simple fatty liver, non-alcoholic steatohepatitis (NASH), steatohepatitis with fibrosis, etc., and the non-alcoholic fatty liver disease mentioned herein may encompass all of the above-described diseases.

These non-alcoholic fatty liver diseases mostly involve insulin resistance, obesity, diabetes and/or hyperlipidemia. In the case of involving such complications, these diseases must be treated first. A principle for treatment of non-alcoholic fatty liver diseases is to improve life habits such as diet, exercise, etc. However, it is difficult, in fact, to exactly practice such improved life habits.

Since non-alcoholic steatohepatitis (NASH) has high likelihood to advance to cirrhosis or hepatocellular carcinoma, more aggressive treatment with medication is required. Although treatments to improve oxidation stress or insulin resistance which is considered to be significant in pathological condition incident/progression of non-alcoholic steatohepatitis have been attempted, a novel treatment method with definitely established scientific grounds has yet to be discovered.

Protectin DX (PDX) is an isomer of protectin/neuroprotectin Dl, which is derived from co-3 fatty acid DHA (docosahexaenoic acid) having anti-inflammatory and anti-diabetic properties. It has been reported that PDX inhibits replication of influenza virus through RNA export system. However, PDX effects upon ER stress and high fat diet (HFD)-derived liver diseases have not yet been reported.

In this regard, the present inventors have firstly researched effects of PDX upon lipid metabolism and TG accumulation in a hyperlipidemia status, and have found a PDX-mediated protective mechanism against palmitate-induced ER stress and fatty liver in HepG2 liver cells. Further, according to experiments on animal models to study influence of PDX upon ER stress, fatty liver and hyperlipidemia, the inventors have firstly disclosed that PDX is a drug capable of being used as a therapeutic agent for hyperlipidemia and ER stress-mediated diseases.

### SUMMARY OF THE INVENTION

Exemplary embodiments provide a method for treating hyperlipidemia or fatty liver disease in a subject, the method comprising administering an effective amount of a composition comprising protectin DX of the following Formula 1 or a pharmaceutically acceptable salt thereof as an active ingredient to a subject in need thereof:

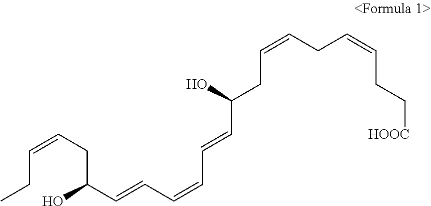 [SMILES: CC/C=C\C[C@H](O)/C=C/C=C\C=C\[C@@H](O)C/C=C\C/C=C\CCC(=O)O]

Exemplary embodiments provide the above mentioned method wherein the protectin DX has effects of decreasing a content of triglyceride in liver tissues.

Another exemplary embodiments provide the above mentioned method wherein the composition is a pharmaceutical or food composition.

Another exemplary embodiments provide the above mentioned method wherein the fatty liver disease is selected from a group consisting of hepatitis, cirrhosis, hepatocellular carcinoma, alcoholic fatty liver, non-alcoholic fatty liver, nutritional fatty liver, starvation-based fatty liver and hepatomegaly.

Exemplary embodiments provide a method for protecting liver in a subject, the method comprising administering an effective amount of a composition comprising protectin DX of the following Formula 1 or a pharmaceutically acceptable salt thereof as an active ingredient to a subject in need thereof:

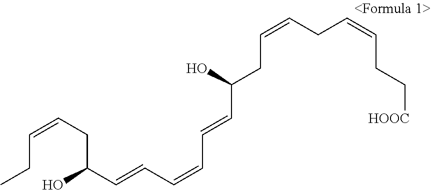 [SMILES: CC/C=C\C[C@H](O)/C=C/C=C\C=C\[C@@H](O)C/C=C\C/C=C\CCC(=O)O]

Another exemplary embodiments provide the above mentioned method wherein the composition is a pharmaceutical or food composition.

### BRIEF DESCRIPTION OF THE DRAWINGS

FIGS. 1A, 1B and 1C show experimental results to support and identify that PDX reduces expression of genes acting for palmitate-induced TG accumulation and lipid generation, respectively. In particular, FIG. 1A shows a result of quantifying TG accumulation in HepG2 cells by Oil-Red-O staining the cells for 24 hours and then extracting TG with isopropyl alcohol; FIG. 1B shows a result of determining expression levels of processed SREBP1, FAS and SCD1 in HepG2 cells; and FIG. 1C shows a result of determining phosphorylation and expression levels of IRE-1, eIF2α and CHOP as ER stress markers (as compared to a control group, ***P<0.001 and **P<0.01; as compared to palmitate-treated group, !!!P<0.001, !!P<0.01 and !P<0.05).

FIGS. 2A, 2B, and 2C show experimental results to demonstrate the effects of PDX upon palmitate-induced ER stress and TG accumulation in HepG2 cells, respectively. In particular, FIG. 2A shows a result of western-blot assay of phosphorylation and expression of ER stress marker; FIG. 2B shows a result of quantifying TG accumulation in HepG2 cells by Oil-Red-O staining the cells for 24 hours and then extracting TG with isopropyl alcohol; FIG. 2C shows a result of western-blot assay of expression of processed SREBP1, FAS and SCD1, which are adipogenesis-related genes in HepG2 cells (as compared to a control group or a scramble control group, ***P<0.001, **P<0.01 and *P<0.05; as compared to palmitate- or PDX-treated group, ***P<0.001, **P<0.01 and *P<0.05).

FIGS. 3A and 3B show increase in ORP150 expression through FOXO1 deacetylation by PDX. In particular, FIG. 3A shows a result of western-blot assay of ORP150 in HepG2 cells transfected with scramble siRNA or siFOXO1 in the presence of 2 μM PDX for 24 hours; and FIG. 3B shows a result of western-blot assay of FOXO1 acetylation in HepG2 cells transfected with scramble siRNA or siAMPK in the presence of 0 to 2 μM PDX for 24 hours (as compared to a scramble control group, ***P<0.001 and **P<0.01; as compared to PDX-treated group, !!!P<0.001 and !P<0.05).

FIGS. 4A and 4B show results of inhibiting palmitic acid-induced TG accumulation through over-expression of ORP150 in liver cells, respectively. In particular, FIG. 4A shows a result of western-blot assay of processed SREBP1 expression in HepG2 cells treated with 200 μM palmitate and/or 0 to 4 μg of ORP150 for 24 hours; FIG. 4B shows a result of quantification of HepG2 cells treated with palmitate for 24 hours by Oil-Red-O staining the cells and then extracting TG with isopropyl alcohol (as compared to a vehicle (control) group, ***P<0.001; as compared to palmitate-treated group, !!!P<0.001 and !P<0.05).

FIGS. 5A, 5B, 5C, 5D, and 5E illustrate improvement in hepatic steatosis and ORP150 expression by systemic administration of PDX, respectively. In particular, FIG. 5A shows a result of H&E and Oil-Red-O staining in livers of normal diet (ND), high fat diet (HFD) and HFD+PDX administered mice, respectively, in the left view, and a result of determining TG accumulation in the liver by means of TG analysis kit in the right view; FIG. 5B shows a result of western-blot assay of SREBP1 (treatment), FAS and SCD1 expression in the livers of the experimental mice; FIG. 5C shows a result of western-blot assay of phosphorylation and expression of IRE-1, elF2a and CHO, which correspond to ER stress markers; FIG. 5D shows a result of western-blot assay of ORP150 expression; and FIG. 5E shows a result of determining adiponectin expression level in the serum of each of the experimental mice (as compared to ND administered group, ***P<0.001 and **P<0.01; as compared to HFD group, !!!P<0.001, !!P<0.01 and !P<0.05).

FIGS. 6A, 6B, 6C and 6D show results of measuring body weights, daily energy intakes, liver weights and epididymal fat contents of normal diet (ND), high fat diet (HFD) and HFD+PDX administered mice, respectively (as compared to ND control group, ***P<0.001 and **P<0.01; as compared to HFD group, !!P<0.01 and !P<0.05).

### DETAILED DESCRIPTION OF THE ILLUSTRATED EMBODIMENTS

Hereinafter, the present invention will be described in more detail.

In accordance with an aspect of the present invention, there is provided a pharmaceutical composition for prevention or treatment of hyperlipidemia or fatty liver disease, which includes protectin DX represented by Formula 1 below or pharmaceutically acceptable salts thereof as an active ingredient:

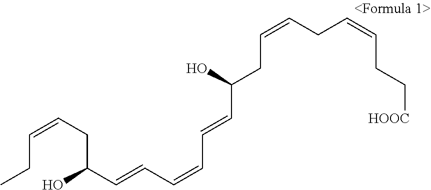 [SMILES: CC/C=C\C[C@H](O)/C=C/C=C\C=C\[C@@H](O)C/C=C\C/C=C\CCC(=O)O]

Protectin DX is also known as “10S,17S-dihydroxy-docosa-4Z,7Z,11E,13Z,15E,19Z-hexaeonic acid”, which can be isolated and purified from natural sources, is commercially available or may be produced by any conventional chemical synthesis process known in the art.

The protectin DX according to the present invention may be used by itself or in the form of a pharmaceutically acceptable salt.

A “pharmaceutically acceptable salt” used herein means a non-toxic composition that generally does not cause allergic reaction or similar reaction and other reactions similar thereto when administered to humans. Such a salt as described above may include an acid-addition salt formed using pharmaceutically acceptable free acids. Such free acids used herein may include organic acids and inorganic acids. The organic acids may include, without being particularly limited to, citric acid, acetic acid, lactic acid, tartaric acid, maleic acid, fumaric acid, formic acid, propionic acid, oxalic acid, trifluoroacetic acid, benzoic acid, gluconic acid, metaesulfonic acid, glycolic acid, succinic acid, 4-toluene sulfonic acid, glutamic acid and aspartic acid. Further, the inorganic acids may include, without being particularly limited to, hydrochloric acid, bromic acid, sulfuric acid and phosphoric acid.

The pharmaceutical composition according to the present invention may include the above novel compound alone or may be formulated in a suitable form along with any pharmaceutically acceptable carrier. Also, any excipient or diluent may be further included. The carrier may include all kinds of solvents, dispersion media, oil-in-water or water-in-oil emulsions, aqueous compositions, liposomes, microbeads and microsome.

The pharmaceutically acceptable carriers may further include, for example, a carrier for oral administration or a carrier for parenteral administration. The carrier for oral administration may include, for example, lactose, starch, cellulose derivatives, magnesium stearate, stearic acid, etc. Further, various drug delivery materials used for oral administration may also be included. Meanwhile, the carrier for parenteral administration may include water, oil, saline solution, aqueous glucose, glycol, etc. Moreover, stabilizers and preservatives may also be included. Further, a suitable stabilizer may include, for example, an antioxidant such as sodium hydrosulfite, sodium sulfite or ascorbic acid. A suitable preservative may include, for example, benzalkonium chloride, methyl- or propyl-paraben and chlorobutanol. The pharmaceutical composition of the present invention may further include lubricants, sweeteners, flavoring agents, emulsifiers, suspending agents, etc., in addition to the above components. For reference, other pharmaceutically acceptable carriers or formulating agents have been described in the following documents (Remington's Pharmaceutical Sciences, 19th ed., Mack Publishing Company, Easton, Pa., 1995).

The composition of the present invention may be administrable to mammal animals including humans using any method. For instance, the composition may be administered orally or parenterally. Parenteral administration may include, without being particularly limited to, intravenous, intramuscular, intra-arterial, intradural, intracardiac, percutaneous, subcutaneous, intraperitoneal, intranasal, intestinal, local, sublingual or intra-rectal administration.

The pharmaceutical composition of the present invention may be prepared into a formulation for oral or parenteral administration.

In the case of the formulation for oral administration, the pharmaceutical composition of the present invention may be formulated into a powder, granules, tablets, pills, sugar-coated tablets, capsules, liquid, gel, syrup, slurry, suspension, etc. by any conventional method known in the art. For instance, the oral formulation may be produced in the form of a tablet or sugar-coated tablet by mixing an active ingredient with a solid excipient, grinding the same, and adding a suitable adjuvant thereto to prepare a granular mixture. Such suitable excipient may include, for example: sugars such as lactose, dextrose, sucrose, sorbitol, mannitol, xylitol, erythritol and maltitol, starches such as corn starch, wheat starch, rice starch and potato starch; celluloses such as cellulose, methyl cellulose, sodium carboxymethyl cellulose and hydroxypropylmethyl-cellulose, etc.; fillers such as gelatin, polyvinyl pyrrolidone, etc. Occasionally, cross-linked polyvinyl pyrrolidone, agar, alginic acid or sodium alginate, etc. may be added as a disintegrating agent. Moreover, the pharmaceutical composition may further include anti-coagulating agents, lubricants, wetting agents, flavors, emulsifiers, preservatives, or the like.

A formulation for parenteral administration may be produced in the form of an injection, cream, lotion, external ointment, oil, moisturizer, gel, aerosol, nasal inhaler, etc. by any conventional method known in the art. Such formulations have been described in prescription documents commonly known in the art (Remington's Pharmaceutical Science, 19th ed., Mack Publishing Company, Easton, Pa., 1995).

A total effective amount of the composition according to the present invention may be a single dose to be administered to a patient, or may be used in multiple-doses according to a fractioned treatment protocol for long term administration. The pharmaceutical composition of the present invention may be used in different contents in term of active ingredient depending upon severity of disease. Preferably, a total effective dose of the pharmaceutical composition according to the present invention may range from 0.01 μg to 10,000 mg, most preferably, 0.1 μg to 500 mg per 1 kg of body weight of patient in a day. However, the above-defined effective dose of the pharmaceutical composition to be administered is generally determined in consideration of not only formulation method, administration route and number of treatments, but also various parameters such as age, body weight, health condition or gender of a patient, severity of disease, diet, excretion rate, etc., and therefore, desired effective dose of the composition may be appropriately determined by a person who has ordinary knowledge in the art to which the present invention pertains (‘the person skilled in the art’). That is, the pharmaceutical composition according to the present invention is not particularly limited in terms of formulation, administration route and administration method so far as desired effects of the present invention could be accomplished.

Further, the pharmaceutical composition of the present invention may be administered in combination with known compounds having effects of preventing or treating hyperlipidemia or fatty liver disease.

“Hyperlipidemia” used herein refers to a disease that occurs due to a high fat level in blood since metabolism of lipids such as cholesterol is not normally performed. More particularly, hyperlipidemia means high-incidence hypercholesteroldemia that exhibits increase in lipid components such as triglyceride, LDL-cholesterol, phospholipids, free fatty acid, etc. in blood.

“Fatty liver disease” used herein is also called fatty liver and refers to a disease caused by abnormal fat (triglyceride) accumulation in liver cells, leading to liver dysfunction. Fatty liver disease may be selected from the group consisting of hepatitis, cirrhosis, hepatocellular carcinoma, alcoholic fatty liver, non-alcoholic fatty liver, nutritional fatty liver, starvation-based fatty liver and hepatomegaly.

The PDX-containing composition of the present invention has activity to prevent, treat or improve the diseases described above. That is, the composition may inhibit the progress of fatty liver disease and treat the disease, and may exhibit effects of preventing occurrence of the same. In particular, the composition may reduce a content of lipids in blood serum and liver cells and thus exhibit activity of preventing and treating or improving hyperlipidemia and fatty liver disease. Further, the composition may normalize activity of a liver function indicator enzyme and thus improve lipid accumulation in liver cells, thereby preventing occurrence of fatty liver, inhibiting the progress of the same and improving the disease condition.

One example of the present invention has demonstrated that PDX has excellent inhibitory activities against SFEBP1, FAS and SCD1, which are known as adipogenic genes. In particular, SREBP is a transcription gene in basic helix loop helix leucine zipper superfamily, which is originally present in the endoplasmic reticulum and, when activated, moves to the nucleus through a processing stage. This gene is known to control expression of genes relevant to the synthesis of fatty acid and triglyceride. It could be seen that processed SREBP-1 expression is mostly predominant in the liver. Representative genes controlled by SREBP-1 may include, for example, acetyl-CoA carboxylase (ACC), fatty acid synthase (FAS), stearoyl-CoA desaturase 1 (SCD1), glycerol-3-phosphate transferase (GPAT), etc. These genes act as an enzyme in a process wherein acetyl-CoA is converted into fatty acid, which in turn synthesizes triglyceride.

A further example of the present invention has demonstrated that accumulation of triglyceride (TG) in liver cells is significantly inhibited by PDX administration, and PDX induces expression of ORP150 to thus inhibit ER stress.

Another example of the present invention has demonstrated that an adiponectin level decreased by HFD diet is increased again by PDX administration.

Another example of the present invention has demonstrated that, when PDX is also administered to HFD diet mice, a body weight, a liver weight and a weight of epididymis of the mouse are significantly reduced.

Still a further example of the present invention has been conducted such that liver cells were collected from a group of experimental animals according to the present invention and analyzed through hematoxylin-eosin (H&E) staining and Oil-Red-O staining. As a result of the analysis, it could be seen that a rate of intracellular fat deposit is reduced in PDX administration group compared to a control group, and hepatic fibrosis occurs very little.

As demonstrated in the examples of the present invention, it is understood that PDX may be effectively used in preventing or treating hyperlipidemia and fatty liver disease.

Moreover, there is provided a food composition for prevention or improvement of hyperlipidemia or fatty liver disease, which includes protectin DX represented by Formula 1 or its salt according to the present invention as an active ingredient.

The food composition of the present invention may include all types of products such as functional foods, nutritional supplements, health foods and food additives.

The above types of food compositions may be manufactured in diverse forms by any conventional method known in the art.

For instance, the health food may be manufactured in the form of a tea, juice and/or beverage for drinking purpose using a novel compound of the present invention, or may be manufactured in a form of granules, capsules and/or powders. Further, materials or active ingredients known to be effective for preventing and improving degenerative brain diseases may be further mixed with the novel compound to prepare a composition according to the present invention.

Further, the functional food may be manufactured by adding the novel compound or its derivatives or pharmaceutically acceptable salts thereof to beverages (including alcoholic drinks), fruits and processed food thereof (e.g., canned fruit, bottled food, jam, marmalade, etc.), fish, meat and processed food thereof (e.g., ham, sausage, corn beef, etc.), bread and noodles (e.g., Japanese-style noodles, buckwheat noodles, instant noodles, spaghetti, macaroni, etc.), juice, various drinks, cookie, taffy, dairy products (e.g., butter, cheese, etc.), edible vegetable fat and oil, margarine, vegetable protein, retortable pouch food, frozen food, condiments (e.g., soy paste, soy sauce, other sauces, etc.), or the like.

A preferable content of the novel compound described above in the food composition of the present invention is not particularly limited but may range from 0.001 to 30% by weight (‘wt. %’), more preferably, 0.01 to 20 wt. % to the finally manufactured food.

Further, in order to use the novel compound of the present invention in the form of a food additive, the compound may be prepared and used in the form of a powder or a concentrated solution.

Further, the present invention provides a pharmaceutical composition for protection of the liver, which includes protectin DX represented by Formula 1 or pharmaceutically acceptable salts thereof as an active ingredient.

Liver protective effects of protectin DX of the present invention may be considered to be obtained as a result of inhibiting triglyceride accumulation in liver cells and/or tissues, inhibiting expression of genes in relation to ER stress and lipid generation and inhibiting hepatic fibrosis according to DX treatment.

Accordingly, “use for liver protection” used herein refers to all uses for protecting liver cells or tissues, which in turn protects both healthy and injured livers without particular limitation. The composition for protection of the liver may refer to a composition for inhibiting, preventing or improving liver injury, and further accomplish effects of preventing or treating liver diseases.

The protectin DX of the present invention may be used by itself or in the form of a pharmaceutically acceptable salt. A detailed description thereof will be substantially the same as the above.

Moreover, the pharmaceutical composition of the present invention may be administered in combination with known compound having liver protective effects.

Further, the present invention provides a food composition for liver protection, which includes protectin DX represented by Formula 1 or a salt thereof as an active ingredient.

The food composition of the present invention may include all types of products such as functional foods, nutritional supplements, health foods and food additives. A detailed description thereof will be substantially the same as the above.

Some embodiments according to the present invention provide a method for treating hyperlipidemia or fatty liver disease in a subject, the method comprising administering an effective amount of a composition comprising protectin DX of the following Formula 1 or a pharmaceutically acceptable salt thereof as an active ingredient to a subject in need thereof:

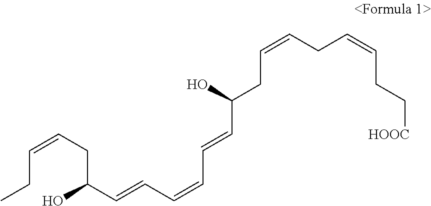 [SMILES: CC/C=C\C[C@H](O)/C=C/C=C\C=C\[C@@H](O)C/C=C\C/C=C\CCC(=O)O]

The term “effective amount” of the present invention refers to an amount that, when administered to a subject, leads to the improvement, alleviation, treatment, or prevention of hyperlipidemia or fatty liver disease.

As used herein, the term “treatment” or “treating” refers to inhibition of disease development, inhibition of recurrence, alleviation of symptoms, reduction of direct or indirect pathological consequences of disease, a reduction in the rate of disease progression, an improvement in the disease state, an improvement, or alleviation.

Another embodiments according to the present invention provide a method for protecting liver in a subject, the method comprising administering an effective amount of a composition comprising protectin DX of the following Formula 1 or a pharmaceutically acceptable salt thereof as an active ingredient to a subject in need thereof:

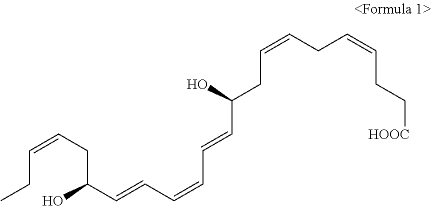 [SMILES: CC/C=C\C[C@H](O)/C=C/C=C\C=C\[C@@H](O)C/C=C\C/C=C\CCC(=O)O]

As used herein, the term “subject” may be an animal, preferably a mammal including humans and livestock, animal-derived cells, tissues, or organs. The subject may be a patient needed for treatment.

Therefore, the present invention relates to novel uses of protectin DX represented by Formula 1 and, in particular, provides a composition for prevention and treatment of hyperlipidemia or fatty liver disease, which includes protectin DX as an active ingredient. The composition including protectin DX according to the present invention exhibits effects of increasing ORP150 expression to reduce ER stress and reducing triglyceride accumulation in liver cells and a weight of the liver. Further, the composition may also reduce a level of adiponectin in blood serum of HFD diet mouse, thereby being effectively used for prevention and treatment of the diseases described above.

Hereinafter, the present invention will be described in detail.

However, the following examples are proposed only to illustrate the present invention and subject matters of the present invention are not particularly limited to the examples.

### Preparation of Experiments

Cell Culture, Reagent and Antibody

Human liver cells, that is, HepG2 cells (ATCC, Manassas, Va., USA) were cultured in high-glucose Dulbecco's modified eagle medium (DMEM, Invitrogen, Carlsbad, Calif., USA), which includes 10% fetal bovine serum (FBS, Invitrogen), 100 units/mL of penicillin and 100 μg/ml of streptomycin. The cells were incubated at 37° C. under a condition of 5% CO₂.

Mycoplasma was not detected in HepG2 cells. PDX (Cayman Chemical, Ann Arbor, Mich., USA) was dissolved in ethanol. Sodium palmitate (Sigma, St. Louis, Mo., USA) was mixed with 2% BSA (fatty acid free level; Sigma) in DMEM. A final concentration of ethanol did not affect the survival of cells. In all experiments, the cells were treated with palmitate-BSA and PDX for 24 hours, while 2% BSA-ethanol was used as a control group.

Anti-phospho IRE-1 (1:1000), anti-IRE-1 (1:2500), anti-phospho eIF2α (1:1000), anti-eIF2α (1:1000), anti-CHOP (1:1000), anti-ORP150 (1:2500) and anti-P62 (1:2500) were purchased from Cell Signaling (Beverly, Mass., USA). Anti-LC3 (1:1000) was purchased from Novus Biologicals (Littleton, Colo., USA). Further, anti-SREBP1 (1:2500), anti-FAS (1:2500), anti-SCD1 (1:2500), anti-GPR78 (1:2500), anti-HSP47 (1:2500), anti-Calnexin (1:2500), anti-HSP70 and anti-beta actin (1:5000) were purchased from Santa Cruz Biotechnology (Santa Cruz, Calif., USA).

Maintenance of Experimental Animals

The present study has received the approval of the Institutional Animal Review Committee (Institutional Animal Care and Use Committee in Korea University, Seoul, Korea). The animal experiment was conducted according to the Guide for Care and Use of Laboratory Animals (NH publication, 8^{th} Ed., 2011). A control group and a group of male C57BL/6J(B6) mice at 8 weeks of age were subjected to a normal diet (ND, Brogaarden, Gentofte, Denmark) and a high fat diet (HFD, Research Diets, New Brunswick, N.J., USA) for 8 weeks, respectively. The HFD (high fat diet) group was provided with intra-peritoneal injection of PDX for 8 weeks (1 μg/mice/day). An ethanol injection group was used as a control group.

Western-Blot Assay

After collecting HepG2 cells, protein was extracted from the cells with a buffer (PRO-PREF; Intron Biotechnology, Seoul, Korea) at 4° C. for 1 hour. A protein sample (35 μg) was applied to 12% SDS-PAGE, transferred to a nitrocellulose film (Amersham Bioscience, Westborough, Mass., USA), detected with a primary antibody, followed by probing with a secondary antibody and another secondary antibody conjugated with horseradish oxidase (Santa Cruz Biotechnology). The sample was detected by ECL kit.

Immuno-Precipitation

Whole protein of HepG2 cell was extracted using an immune-precipitation buffer (IP buffer: 50 mM Tris-HCl, pH 7.8, 150 mM NaCl, 1% IGEPAL CA630) and then diluted to a concentration of 1 mg/mL. A polyclonal antibody to FOXO1 (Santa Cruz Biotechnology) diluted 1:150 was added to the mixture and the sample was incubated at 4° C. overnight. After incubation, each of the samples was provided with 50 μL of a protein A/G-Sepharose bead suspension (Santa Cruz Biotechnology), followed by gently mixing the same at 4° C. for 1 hour. The treated samples were subjected to centrifugation at 12,000 rpm for 30 seconds; and the beads were washed in IP buffer three times. The isolated beads were suspended again in 1×SDS-PAGE loading buffer, heated at 95° C. for 5 minutes, followed by vortexing and flash-centrifugation. The obtained supernatant was loaded on 12% SDS-polyacrylamide gel for electrophoresis and western-blot assay.

Transient Transfection for Genetic Silencing or Over-Expression

Under 70% comfluency, 0 to 20 nmol/L low-interference (si)RNA oligonucleotide to ORP150 purchased from Santa Cruz Biotechnology was transfected to inhibit gene expression. Scramble siRNA was used as a control group. 2 or 4 μg of pCMV3-ORP150 (Sino Biological, Beijing, China) was transiently transfected, leading to over-expression of ORP150. pCMV3 empty-vector was used as a control group. According to instruction of the manufacturer, transfection was executed using lipofectamine 2000 (Invitrogen).

Histological Assay

HepG2 cells and a cut section of the liver in a mouse were stained by Oil-Red-O staining method, in order to determine cellular triglyceride such as triglyceride, that is, TG. The liver cells were fixed with 10% formalin for 40 minutes, and then stained with Oil-Red-O solution (Sigma) at 37° C. for 1 hour. A content of Oil-Red-O stained TG was quantified by adding isopropanol to each of the samples. The mixture was gently stirred at 25° C. for 8 minutes. Lastly, 100 μl of isopropanol-extracted sample was analyzed by a spectrophotometer at 510 nm.

TG Measurement

Total lipids were extracted using a mixture of 2:1 chloroform:methanol (2:1, v/v). An organic layer was dissolved in 60% methanol immediately after drying the same. The extracted TG was measured by a colormetric TG analysis kit according to instructions of the manufacturer (Biovision, Milpitas, Calif., USA).

Statistical Analysis

All analyses were performed with SPSS/PC statistical program (Windows version 12.0; SPSS, Chicago, Ill., USA). Analyzed results were reported as multiples of the highest value (means±s.d.). Every in vitro experiment was carried out three times. For statistical analysis, Student's t test or two-way ANOVA was used.

### Experimental Results

PDX Effects of Inhibiting Palmitate-Induced TG Accumulation Caused by ER Stress in Liver Cells

In the presence of 200 μM palmitate and PDX (0 to 2 μm), HepG2 cells were subjected to Oil-Red-O staining for 24 hours, while TG accumulation was quantified by isopropyl alcohol extraction.

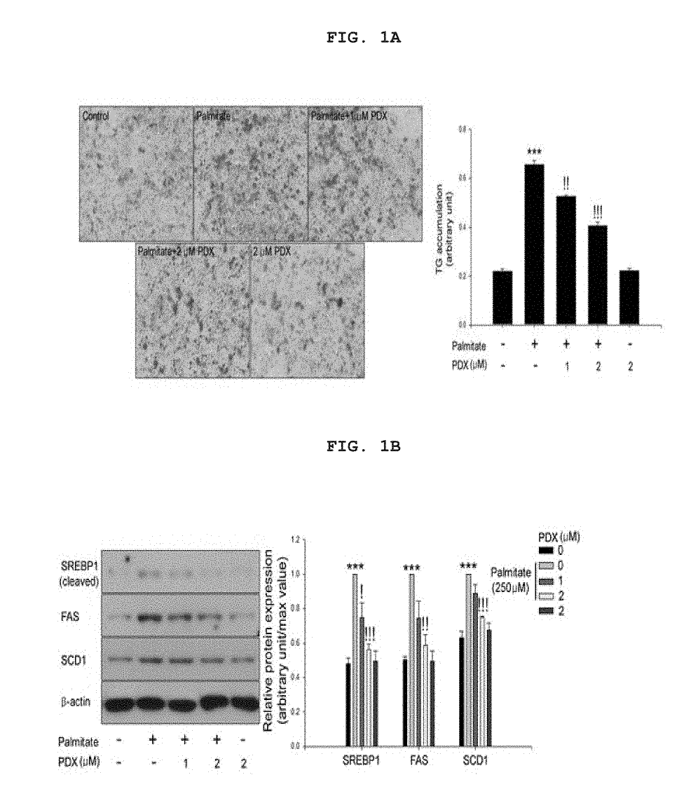

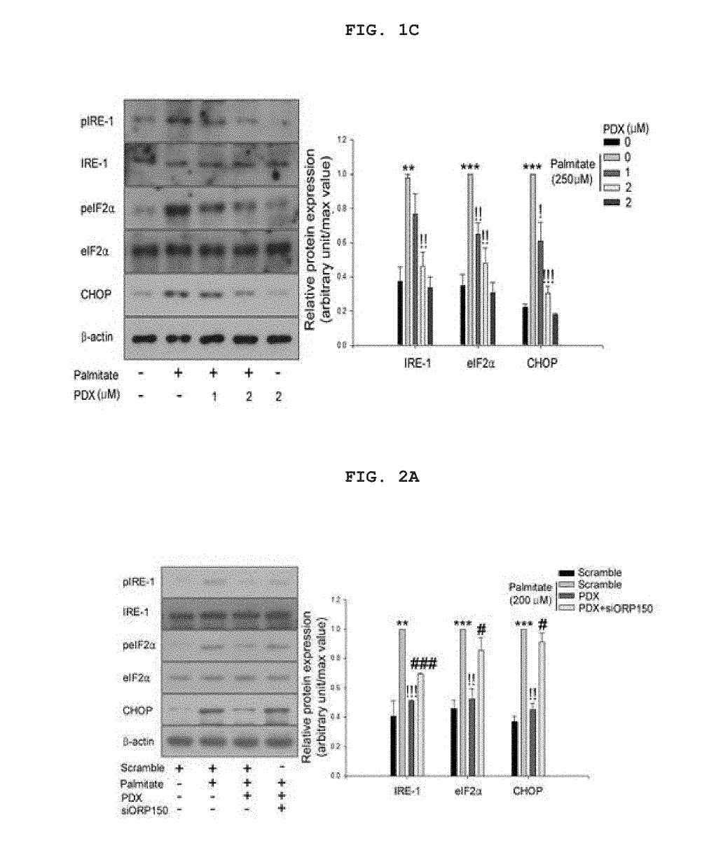

In this regard, as shown in FIG. 1A and FIG. 1B, it could be seen that palmitate-induced TG accumulation and expression of adipogenesis-related genes including SREBP1, FAS and SCD1 in HepG2 cells (liver cells) were inhibited by PDX. These results are obtained by assessing PDX effects upon ER stress caused by palmitate, since palmitate increases ER stress and causes lipid accumulation through SREBP1-mediated route.

Further, as shown in FIG. 1C, when HepG2 cells were treated with PDX, palmitate-induced stress and expression of relevant genes were significantly reduced depending upon PDX administration dose. More particularly, phosphorylation and expression of ER stress markers such as IRE-1, eIF2α and CHOP were observed, and a concentration of such marker was significantly decreased depending upon a dose of PDX.

Effect of Inhibiting ER Stress and TG Accumulation in Liver Cells Through Induction of ORP150 Expression by PDX

The present inventors investigated whether ORP150 is relevant to ER stress and TR accumulation inhibitory effects of PDX.

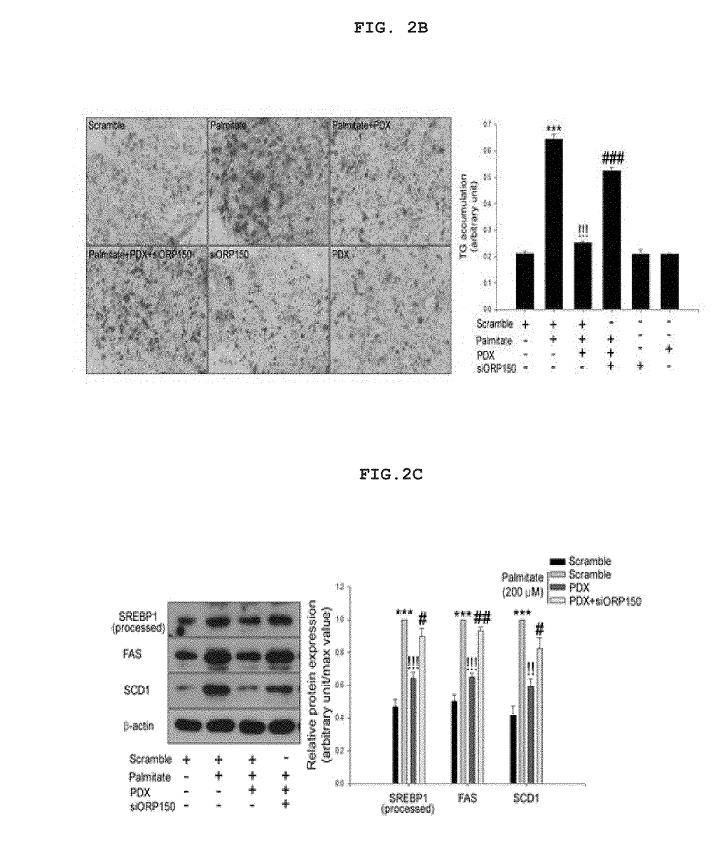

As shown in FIG. 2A to FIG. 2C, silencing of si-RNA-mediated ORP150 has suppressed inhibition of ER stress in HepG2 cells, and therefore, has significantly reduced PDX effects upon TG accumulation induced by palmitate.

More particularly, as shown in FIG. 2A, expression of IRE-1, eIF2α and CHOP, which are ER stress markers, were determined with respect to scramble group/scramble+palmitate-induced group/scramble palmitate-induced PDX administered group/scramble+palmitate-induced+PDX administered+siOPR150 group that achieved silencing of ORP150, respectively. As a result, it was found that PDX reduced the expression of ER stress markers, however, this expression was increased again by siORP150 administration. Accordingly, it is understood that ORP150 may inhibit the expression of ER stress.

FIG. 2B shows a result of measuring TG accumulation with respect to the PDX administered group or the ORP150 silencing group. As shown in FIG. 2B, it could be seen that palmitate-induced TG accumulation was inhibited by PDX administration, and TG accumulation inhibitory effects of PDX were reduced by silencing ORP150.

As shown in FIG. 2C, expression of adipogenesis-related genes such as SREBP1, FAS and SCD1 was inhibited by PDX administration. However, it could be seen that, as a result of silencing ORP150, PDX effects of inhibiting the expression of adipogenesis-related genes were significantly reduced.

Based on the above experimental results, it is understood that PDX may induce ORP150 expression, inhibit ER stress, and reduce palmitate-induced TG accumulation.

Effects of Increasing ORP150 Expression Through FOXO1 Deacetylation by PDX

The present inventors have executed western-blot assay of ORP150 in HepG2 cells transfected with scramble siRNA or siFOXO1 in the presence of 2 μm of PDX for 24 hours.

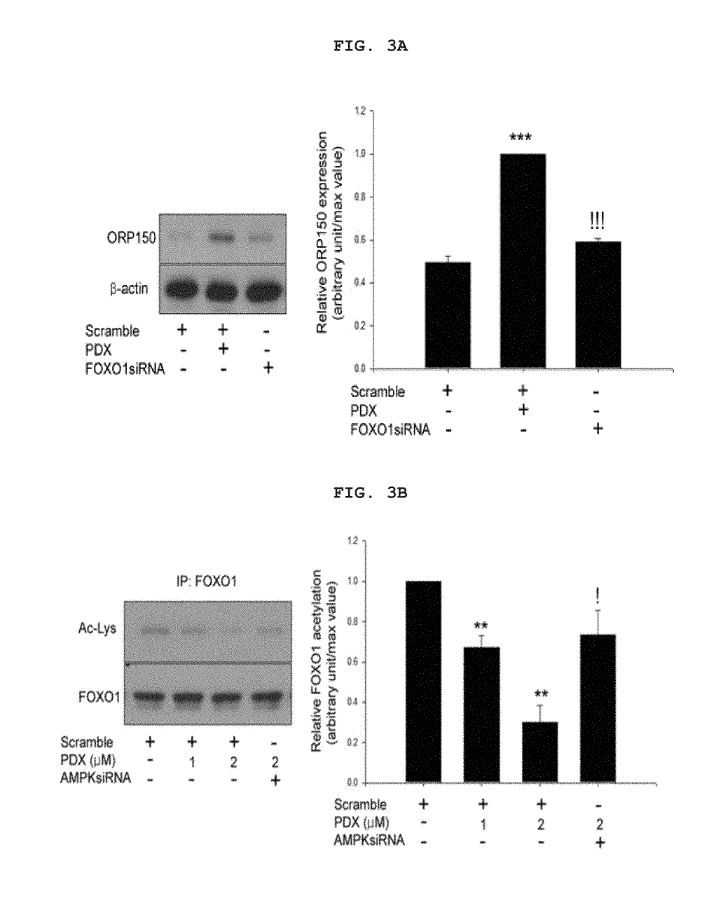

In this regard, as shown in FIG. 3A, it could be seen that ORP150 expression level was increased in PDX-administered liver cells, however, PDX effects of promoting ORP150 expression were inhibited in the liver cells transfected with FOXO1 siRNA.

Further, the present inventors have executed western-blot assay of FOXO1 acetylation in HepG2 cells transfected with scramble siRNA or siAMPK in the presence of 0 to 2 μM PDX for 24 hours.

In this regard, as shown in FIG. 3B, FOXO1 acetylation was reduced depending upon PDX concentration (that is, increase in deacetylation) in the liver cells having received PDX administration, while PDX effects of reducing acetylation in the liver cells transfected with AMPKsiRNA were inhibited.

Based on the above experimental results, it is understood that the present inventive PDX may increase ORP150 expression through FOXO1 deacetylation and, when ORP150 expression is increasing, ER stress may be inhibited to thus inhibit accumulation of triglyceride.

Effects of Inhibiting Palmitic Acid-Induced TG Accumulation Through ORP150 Over-Expression

The present inventors have executed western-blot assay of SREBP1 expression in HepG2 cells that were treated with 200 μM palmitate and/or with 0 to 4 μg of ORP150 for 24 hours.

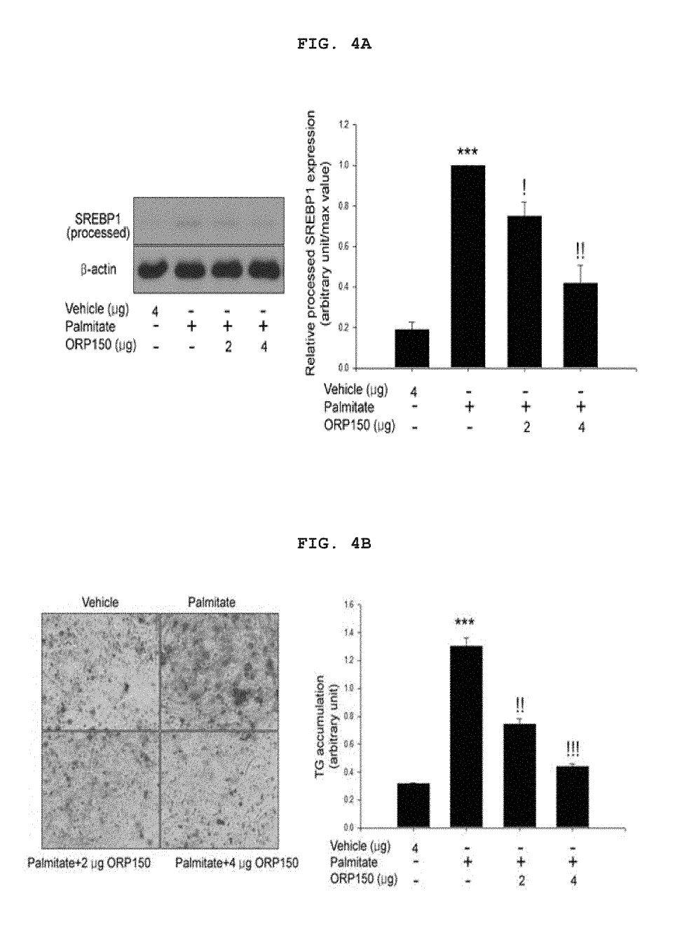

In this regard, as shown in FIG. 4A, SREBP1 expression induced by palmitate was inhibited depending upon a concentration of ORP150 used for treatment.

Further, the present inventors have executed Oil-Red-O staining with respect to palmitate-treated HepG2 cells for 24 hours. TG accumulation was quantified by extraction using isopropyl alcohol.

As shown in FIG. 4B, it could be seen that TG accumulation was significantly reduced depending upon a concentration of ORP150 used for treatment.

Based on the above experimental results, it is understood that ORP150 may inhibit accumulation of triglyceride in the liver cells, and this result substantially supports that PDX may increase ORP150 expression and thus may inhibit accumulation of triglyceride in the liver cells.

Hepatic Steatosis Treatment Effects of PDX in HFD Diet Mouse

The present inventors have assessed PDX effects upon lipid accumulation in a mouse. For this purpose, a liver cut section from a mouse was subjected to histological assay and western-blot assay through H&E staining and Oil-Red-O staining, while TG accumulation was measured using a TG analysis kit.

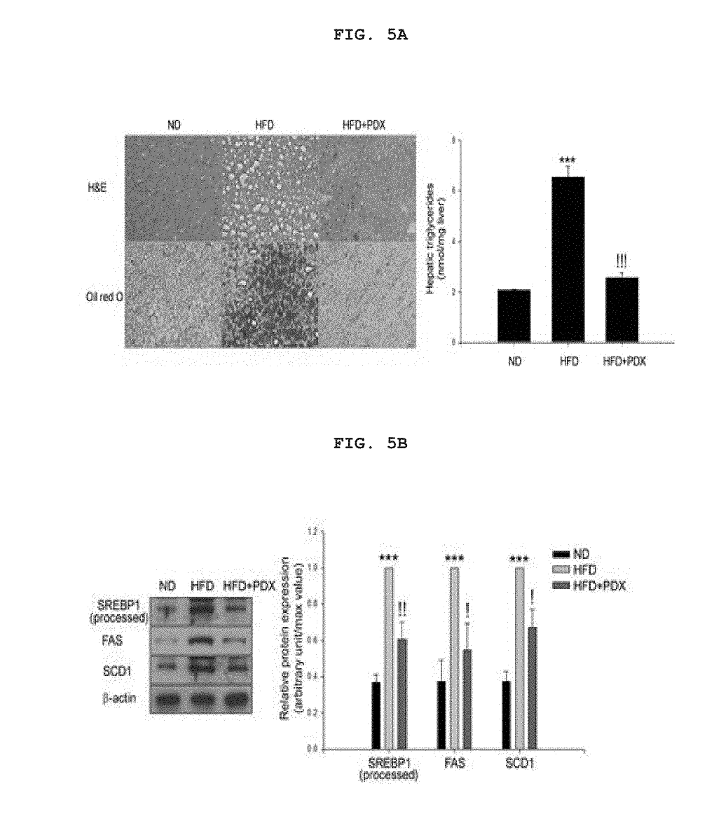

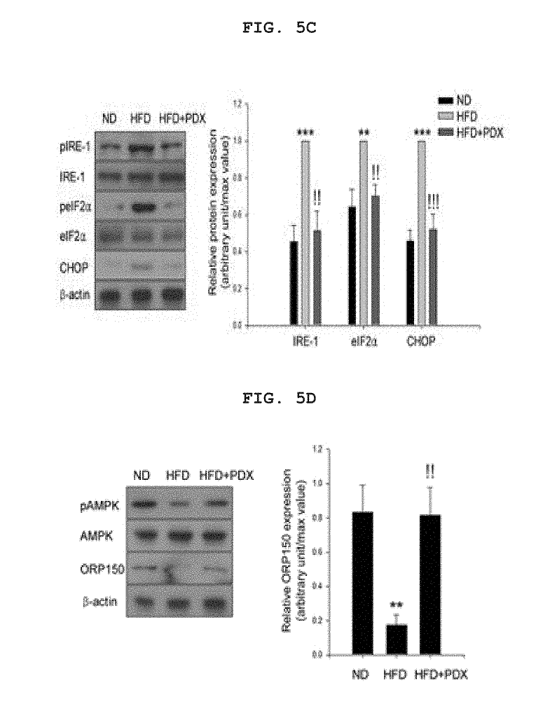

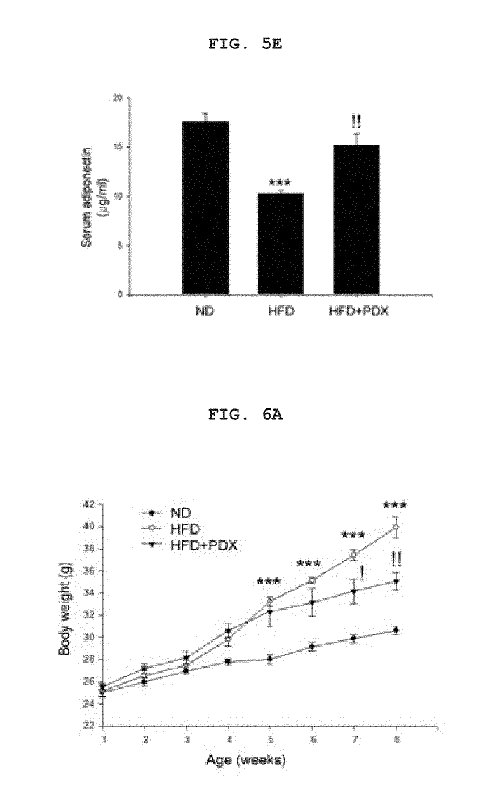

In this regard, as shown in FIG. 5A and FIG. 5B, HFD diet increased TG accumulation in the liver and expression of adipogenesis-related genes such as SREBP1, FAS and SCD1 in the liver. However, PDX administration demonstrated remarkable reversion of such changes as described above. That is, the experimental results demonstrated that accumulation of triglyceride in the liver was considerably reduced and expression of adipogenesis-related genes was also significantly reduced by administering PDX. Further, referring to the left view in FIG. 5A that shows a result of staining the liver cut section, it could be seen that the PDX-administered HFD diet mouse group exhibited a decrease in intracellular fat deposit rate, compared to HFD diet group, and very little hepatic fibrosis.

Further, as shown in FIG. 5C, the liver ER stress markers, that is, IRE-1, eIF2α and CHOP were also inhibited by PDX administration. Further, as shown in FIG. 5D, ORP150 expression suppressed in the liver by HFD was remarkably recovered by PDX treatment.

FIG. 5E shows a result of evaluating an adiponectin value in blood serum, wherein adiponectin is specifically secreted from adipose tissues and generally present with a high concentration in blood, however, it is known that the concentration of adiponectin is decreased by intra-abdominal fat accumulation. PDX administration increased a level of adiponectin in blood serum of the mouse having decreased adiponectin concentration by HFD diet.

PDX Effects of Reducing Body Weight and Liver Weight in Mice

According to the above experimental procedures conducted for 8 weeks, the present inventors measured body weights, daily energy intakes, liver weights and epididymal fat contents, with respect to five mice per group.

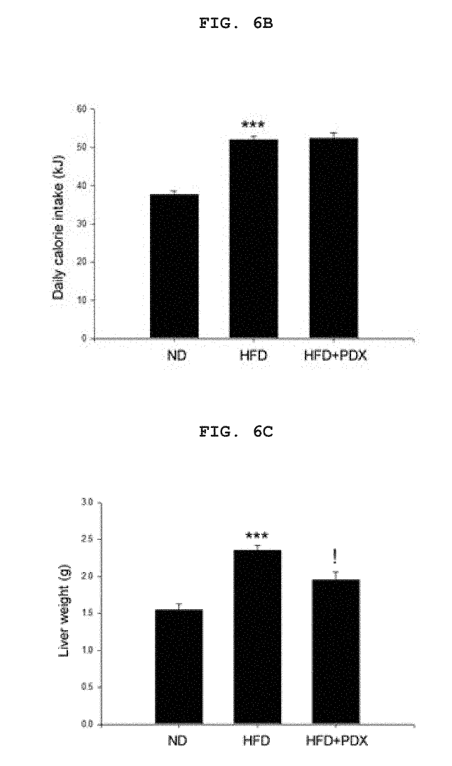

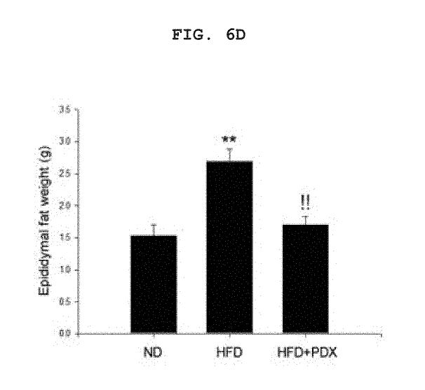

As shown in FIG. 6A, it could be seen that the PDX-administered mice have significantly reduced body weights, compared to the HFD diet mice. Further, as shown in FIGS. 6C and 6D, it could be seen that the liver weights and epididymal fat contents of the PDX-administered mice were also significantly decreased, compared to the HFD diet mice.

As described in the above inventive examples, it is understood that PDX administration effectively reduces accumulation of liver fat, and therefore, PDX may be used as a novel drug for treating fatty liver disease.

As such, the present inventors have demonstrated that protectin DX may inhibit lipid-induced ER stress through induction of ORP150 expression, thereby improving fatty liver and lipid metabolism. Accordingly, protectin DX may be effectively used in a pharmaceutical composition or food composition for preventing or treating hyperlipidemia or fatty liver disease to thus protect the liver, thereby being industrially applicable.

What is claimed is:

1. A method for treating hyperlipidemia or fatty liver disease in a subject, the method comprising administering an effective amount of a composition comprising protectin DX of the following Formula 1 or a pharmaceutically acceptable salt thereof as an active ingredient to a subject in need thereof:

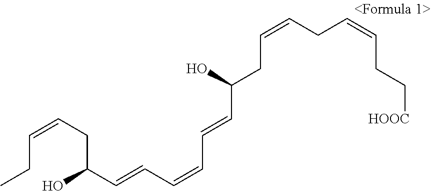 [SMILES: CC/C=C\C[C@H](O)/C=C/C=C\C=C\[C@@H](O)C/C=C\C/C=C\CCC(=O)O]

2. The method of claim 1, wherein the protectin DX has effects of decreasing a content of triglyceride in liver tissues.

3. The method of claim 1, wherein the composition is a pharmaceutical or food composition.

4. The method of claim 1, wherein the fatty liver disease is selected from a group consisting of hepatitis, cirrhosis, hepatocellular carcinoma, alcoholic fatty liver, non-alcoholic fatty liver, nutritional fatty liver, starvation-based fatty liver and hepatomegaly.

5. A method for protecting liver in a subject, the method comprising administering an effective amount of a composition comprising protectin DX of the following Formula 1 or a pharmaceutically acceptable salt thereof as an active ingredient to a subject in need thereof:

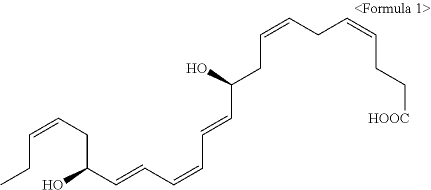 [SMILES: CC/C=C\C[C@H](O)/C=C/C=C\C=C\[C@@H](O)C/C=C\C/C=C\CCC(=O)O]

6. The method of claim 5, wherein the composition is a pharmaceutical or food composition.
